(19) United States
(12) Plant Patent Application Publication
(10) Pub. No.: US 2005/0216990 P1
(43) Pub. Date: Sep. 29, 2005

(54) CALIBRACHOA PLANT NAMED ' SUNBELPISUPU '

(50) PETUNIA HYBRIDA; PETUNIA

(76) Inventors: Takeshi Kanaya, Omihachiman (JP); Yasuyuki Murakami, Gamo-gun (JP)

Correspondence Address: CHRISTIE, PARKER & HALE, LLP, PO BOX 7068, PASADENA, CA, 91109-7068, US

(21) Appl. No.: 10/812,863

(22) Filed: Mar. 29, 2004

(51) Int. Cl. A01H 5/00

(52) U.S. Cl. PLT/263

(57) ABSTRACT

Disclosed herein is a new and distinct variety of Petunia plant having a Spreading growth habit. The Petunia plant has abundant branching, and a great profusion of blooms, the whole plant remaining in bloom for a considerable period of time. The flowers are single and small, the petals having a yellow white color with deep reddish purple vein. The inside color of the corolla throat is vivid yellow and the outside of the corolla tube is light greenish yellow with moderate reddish purple vein. The plant exhibits high resistance to heat, cold, rain and disease.

### BOTANICAL/COMMERCIAL CLASSIFICATION

Petunia hybrida/Petunia Plant

### VARIETAL DENOMINATION

cv. ‘Sunbelpisupu’

### BACKGROUND OF THE VARIETY

The present invention relates to the new variety of Petunia plant originated from crossing of a Petunia hybrid variety called ‘Sunbelki’ as the female parent and ‘W12’ as the male parent.

The Petunia is a very popular plant that is used for flower bedding and potting in the summer season. There are only a few Petunia developed series of small flower petunias, such as, ‘SUNTORY SP-R’ (U.S. Plant Pat. No. 9,557), ‘Sunberubu’ (U.S. Plant Pat. No. 9,754), ‘Sunbelchipi’ (U.S. Plant Pat. No. 10,355) and ‘Sunbelki’ (U.S. Plant Pat. No. 11,558) are of the spreading type, a medium plant height, abundant branching, and a high resistance to heat and rain and disease. However there are only a few varieties having a great profusion of flowers, yellowish white with reddish purple vein, a very small flower size and a high resistance to rain, heat, and disease. Accordingly, this invention was aimed at obtaining a new Petunia variety having yellowish white colored with deep reddish purple vein, and very small flowers, combined with the above features.

Progress

The female parent ‘Sunbelki’ (U.S. Plant Pat. No. 11,558) used in the crossing of ‘Sunbelpisupu’ is a cultivar grown at Yokaichi-shi, Shiga-ken, Japan, having a spreading growth habit with abundant branching. It has small single flowers, the petals having a bright yellow color.

The male parent ‘W12’ (unpatented) used in the crossing of ‘Sunbelpisupu’ is a strain of our breeding lines, having a spreading growth habit with many branches. It has small single flowers, the petals having a white color.

In April 2000, crossing of ‘Sunbelki’ as the female parent and ‘W12’ as the male parent was conducted at Yokaichi-shi, Shiga-ken, Japan. In September 2000, 50 seedlings were obtained from that crossing. These seedlings were grown in pots in glasshouses and were evaluated from April 2001. One seedling was selected in view of its growth habit, flower size and color in September 2001. That seedling was propagated by cutting and a trial was carried out by flower potting and bedding from April to October 2002. The botanical characteristics of that plant were then examined, using similar varieties ‘Sunbelki’ and ‘Sunbelchipi’ for comparison. As a result, it was concluded that this Petunia plant is distinguishable from any other variety, whose existence is known to us, and is uniform and stable in its characteristics. Then the new variety of Petunia plant was named ‘Sunbelpisupu’.

In the following description, the color-cording is in accordance with the Horticultural Colour Chart of The Royal Horticultural Society, London, England (R.H.S. Colour Chart).

### SUMMARY OF THE VARIETY

This new variety is unlike any Petunia commercially available as evidenced by the following unique combinations of characteristics.

1. Spreading growth habit with abundant branching.

2. Having a great profusion of blooms with the entire plant remaining in bloom for a considerable period of time.

3. The flowers are single and small. The petal color is yellow white (R.H.S. 159D) with deep reddish purple (R.H.S. N78A) vein.

4. The plant has a high resistance to rain, cold, heat and disease.

The new variety ‘Sunbelpisupu’ differs from the similar variety ‘Sunbelchipi’ in the following points.

1. The leaf of ‘Sunbelpisupu’ is smaller than that of ‘Sunbelchipi’.

2. The flower of ‘Sunbelpisupu’ is larger than that of ‘Sunbelchipi’.

3. The petal color of ‘Sunbelpisupu’ is yellowish white (R.H.S. 159D) with deep reddish purple (R.H.S. N78A) vein. That of ‘Sunbelchipi’ is vivid purplish red (R.H.S. N57A).

4. The inside color of the corolla throat of ‘Sunbelpisupu’ is vivid yellow (R.H.S. 14B). That of ‘Sunbelchipi’ is strong yellow (R.H.S. 9A).

5. The outside color of the corolla throat of ‘Sunbelpisupu’ is light greenish yellow (R.H.S. 7D) with moderate reddish purple (R.H.S. 79D) vein. That of ‘Sunbelchipi’ is brilliant greenish yellow (R.H.S. 5C).

6. The apex shape of petal of ‘Sunbelpisupu’ is truncate. That of ‘Sunbelchipi’ is obtuse.

The new variety ‘Sunbelpisupu’ differs from the similar variety ‘Sunbelki’ in the following points.

1. The leaf of ‘Sunbelpisupu’ is smaller than that of ‘Sunbelki’.

2. The flower of ‘Sunbelpisupu’ is larger than that of ‘Sunbelki’.

3. The petal color of ‘Sunbelpisupu’ is yellowish white (R.H.S. 159D) with deep reddish purple (R.H.S. N78A) vein. That of ‘Sunbelki’ is bright yellow (R.H.S. 12A).

4. The inside color of the corolla throat of ‘Sunbelpisupu’ is vivid yellow (R.H.S. 14B). That of ‘Sunbelki’ is strong brown (R.H.S. 166B).

5. The outside color of the corolla throat ‘Sunbelpisupu’ is light greenish yellow (R.H.S. 7D) with moderate reddish purple (R.H.S. 79D) vein. That of ‘Sunbelki’ is greenish yellow (R.H.S. 4C) with dark reddish purple (R.H.S. 166A) vein.

The new variety of Petunia Plant ‘Sunbelpisupu’ was asexually reproduced by the use of cuttings at Yokaichi-shi, Shiga-ken, Japan, and homogeneity and stability thereof were confirmed. The instant plant retains its distinctive characteristics and reproduces true to type in successive generations.

### BRIEF DESCRIPTION OF THE PHOTOGRAPH

The depicted plants had been reproduced by the use of cuttings and were photographed during September 2002 while growing outdoors in 24 cm pots at an age of approximately 8 months at Yokaichi-shi, Shiga-ken, Japan.

FIG. 1 illustrates a typical plant of the new variety of Petunia plant ‘Sunbelpisupu’ while growing in a pot.

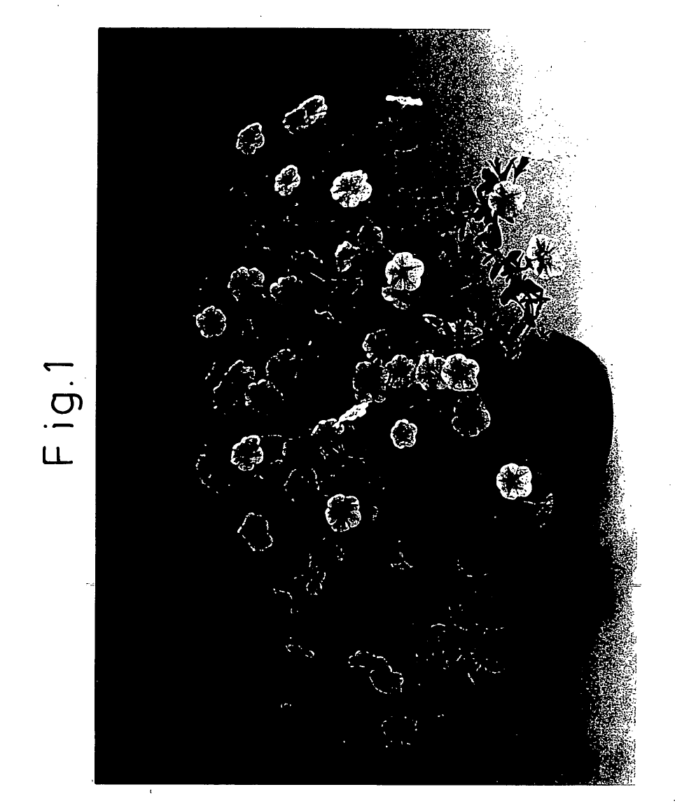

FIG. 2 illustrates a close view of typical blossoms of the new variety of Petunia plant ‘Sunbelpisupu’.

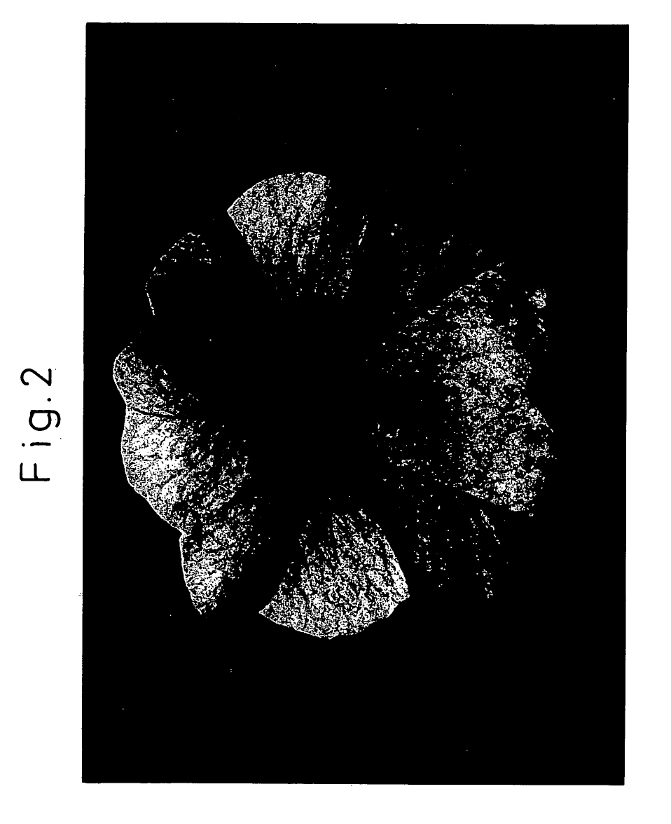

### DESCRIPTION OF THE VARIETY

The botanical characteristics of the new and distinct variety of Petunia plant named ‘Sunbelpisupu’ are as follows when observed during December at Yokaichi-shi, Shiga-ken, Japan, at an age of approximately 11 months.
Plant:
   Growth habit.—Spreading.
   Plant height.—Approximately 21.6 cm.
   Spreading area of plant.—Approximately 59.0 cm.
   Blooming period.—Mid-April to late October in the southern Kanto area, Japan. The plant shape does not change throughout this period.
Stem:
   Length.—Approximately 5.0 cm.
   Thickness.—Approximately 1.6 mm.
   Pubescence.—Normal.
   Branching.—Abundant branching, especially secondary branches are.
   Internode length.—Approximately 1.2 cm.
   Color.—R.H.S. 144B, 200C (strong yellow-green, grayish-brown).
Leaf:
   Whole shape.—Lanceolate. The apex shape is acute, and the base shape is attenuate.
   Length.—Approximately 2.6 cm.
   Width.—Approximately 0.8 cm.
   Color.—Upper and lower side color is R.H.S. 144A (strong yellow green).
   Thickness.—Approximately 0.3 mm.
   Pubescence.—Normal.
Flower:
   Facing direction.—Slanted upward.
   Type.—Single.
   Shape.—Funnel-shape, with five-fissured limb.
   Shape of petal tip.—Truncate.
   Lobation.—Shallow.
   Waving of petal.—Weak.
   Diameter.—Approximately 3.2 cm.
   Color.—Petal; R.H.S. 159D (yellow white) with R.H.S. N78A (deep reddish purple) midvein. Inside color of the corolla throat is vivid yellow (R.H.S. 14B). Outside color of the corolla throat is light greenish yellow (R.H.S. 7D) with moderate reddish purple (R.H.S. 79D) vein.
   Reproductive organs.—1 normal pistil and 5 normal stamens. The stigma is club-shaped and R.H.S. N144D (light yellow green) in coloration. The style is approximately 8 mm in length and R.H.S. 145B (brilliant yellow green) in coloration. The ovary is R.H.S. N144D (light yellow green) in coloration. The stamens commonly are of variable length from approximately 4.0 to 1.0 mm. Pollen is formed in a quantity that is typical of Petunia hybrida and is near R.H.S. 21A (vivid orange yellow) in coloration.
   Fertility.—Fertile, but self-incompatible.
   Peduncle.—Approximately 0.5 mm in diameter and approximately 1.1 cm in length. The texture is smooth.
   Calyx.—Narrow. 5 sepals in fused at the base.
   Seeds.—R.H.S. N186A (strong red) in coloration, approximately 0.6 mm in diameter, and generally round. The quantity is typical of Petunia hybrida.
Physiological and ecological characteristics: High resistance to cold, heat, rain and disease, such as Powdery mildew. The resistance to heat and rain is very strong.

This new variety of Petunia plant is most suitable for flower bedding and potting, particularly in hanging pots or planters. Pinching of old blossoms will enhance the formation of new blossoms.

1. A new and distinct variety of Petunia plant named ‘Sunbelpisupu’, substantially as herein illustrated and described.
